(19) United States
(12) Patent Application Publication
(10) Pub. No.: US 2006/0111620 A1
(43) Pub. Date: May 25, 2006

(54) Providing medical services at a kiosk

(76) Inventors: John R. Squilla, Rochester, NY (US); Kim W. Montz, Webster, NY (US); Donna K. Rankin-Parobek, Honeoye Falls, NY (US); Michael Marou, Bergen, NY (US); Frank Marino, Rochester, NY (US)

Correspondence Address: Mark G. Bocchetti;Patent Legal Staff, Eastman Kodak Company, 343 State Street, Rochester, NY, 14650-2201, US

(21) Appl. No.: 10/995,676

(22) Filed: Nov. 23, 2004

(51) Int. Cl. A61B 5/00

(52) U.S. Cl. 600/300

(57) ABSTRACT

A medical kiosk (10) provides medical services. The medical test to be performed are determined (110). A test kit is dispensed from the medical kiosk (120). The necessary tests are performed. The test kit is decontaminated (172) and the results of the tests are recorded.

### CROSS REFERENCE TO RELATED APPLICATIONS

Reference is made to commonly-assigned copending U.S. patent application Ser. No. ______ (Attorney Docket No. 88970/NAB), filed Nov. 18, 2004, entitled MEDICAL KIOSK WITH MULTIPLE INPUT SOURCES, by Squilla et al.; the disclosure of which is incorporated herein.

### FIELD OF THE INVENTION

This invention relates to the fields of medical testing and medical kiosks, and in particular, to a method of integrating medical testing procedures or delivery and recovery of non-invasive medical test kits in an unassisted kiosk.

### BACKGROUND OF THE INVENTION

Traditionally, many common medical testing and screening procedures have relied on trained professionals performing invasive procedures, such as drawing a blood sample or taking a throat culture. Recently, there has been an increased emphasis on the development of non-invasive testing procedures as alternatives to the existing invasive counterparts. Currently these non-invasive screening techniques analyze breath, urine, saliva, and fecal samples. Additional techniques exist to spectrally or optically analyze a patient's skin or eye (e.g. retina). The present invention teaches an unassisted medical kiosk that performs non-invasive tests: dispenses medical test kits, performs the tests, analyzes the results, and is a self cleaning system that disposes of or decontaminates used test components.

The methods of performing non-invasive tests are well known. Examples include Aerocrine Inc. (www.aerocrine.com), which has developed a technique for measuring the concentration level of nitric oxide (NO) in exhaled human breath to detect asthma related inflammations. SHL Telemedicine Ltd. (www.shahal.co.il) provides a means to measure peak expiratory flow rate for diagnosis and detection of lung disorders. NASA Glenn Research Center (www.grc.nasa.gov) has developed a number of non-invasive techniques to image the eye for detection and diagnosis of cataracts, glaucoma, diabetic retinopathy, macular degeneration, Alzheimer's, and drug screening. Numerous companies have developed techniques to measure glucose levels of diabetics using optical techniques (e.g. University of Texas Medical Branch, Department of Biomedical Engineering Texas A7M) or ionophoresis (Cygnus Inc.). Additionally, Lucid Inc. (www.lucid-tech.com) has developed a laser scanning confocal microscope to detect skin cancer.

There are unassisted medical kiosks in the marketplace that provide basic vital statistics monitoring such as patient heart rate and blood pressure. See LifeClinic at www.lifeclinic.com. U.S. Patent Application Publication No. 2004/0044560 (Giglio et al.) discusses a device to test and output the personal data (fat analysis) of a user to a computer processor. U.S. Pat. No. 6,692,436 (Bluth et al.) teaches a health kiosk that provides blood pressure testing, a health and fitness evaluation, and a medication encyclopedia. Other unassisted kiosks aid a patient in diagnosing a condition by using question and answer scripts to reach a diagnostic conclusion. U.S. Pat. No. 6,641,532 (Iliff) teaches the art of conducting an automated diagnostic session with a patient, using a plurality of disease scripts, a patient medical record, and a disease engine to process the script and route the changes to the medical record. Staffed medical kiosks also exist that provide a nurse to check on certain ailments. See MinuteClinic at www.minuteclinic.com.

All of these medical kiosks provide convenient medical services to consumers with improved accessibility over visits to a doctor's office. However, the unassisted kiosks are limited in their ability to provide comprehensive diagnostic services due to the lack of a means to locally dispense new medical test kits, analyze test results, maintain a sterile environment, and recycle and dispose of used test kits. Assisted kiosks can provide more sophisticated diagnostic testing and screening procedures for patients and can handle disposal and sterilization of used test components. However, they are limited in convenience by their hours of operation, limited number of locations, and staffing costs.

A need exists in the marketplace to further extend the utility of medical kiosks by providing a greater variety of unassisted medical screening and testing procedures in convenient, accessible locations; while ensuring hygiene and patient privacy are maintained.

### SUMMARY OF THE INVENTION

Briefly, according to one aspect of the present invention a medical kiosk provides medical services. The medical test to be performed are determined. A test kit is dispensed from the medical kiosk. The necessary tests are performed. The test kit is decontaminated and the results of the tests are recorded.

The above need is met in the following invention by integrating non-invasive medical testing procedures or medical test kits that are delivered, retrieved, and analyzed in an unassisted medical kiosk. The medical kiosk includes, but is not limited to, monitoring existing conditions, screening and diagnosing a new condition or disease, dispensing medical supplies, and maintaining the hygiene of the kiosk and medical supplies.

The present invention discloses an unassisted medical kiosk that can:
dispense recyclable or disposable non-invasive medical test kits;
dispense basic health care items;
perform test procedures using onboard instrumentation;
analyze test results and provide status information or a diagnosis;
present the results to the consumer and communicate results to a medical professional, insurance company or electronic patient record (EPR);
maintain hygiene by reclaiming test kits, disposing of non-reusable test components, decontaminating itself; and
sterilize reusable test components.

It is important that the medical kiosk be easy to use, with straightforward testing procedures, while retaining security and privacy of the patient's test results and medical information. Continued kiosk usage by the patient will depend on the system's overall hygiene. Dispensing and reclaiming test kits must take place under strictly controlled sanitary conditions.

The invention and its objects and advantages will become more apparent in the detailed description of the preferred embodiment presented below.

### BRIEF DESCRIPTION OF THE DRAWINGS

FIG. 1 is a schematic view of a testing procedures integrated into a medical kiosk.

FIG. 2 is a systems diagram and potential data forms for electronic patient record (EPR) embodiments.

FIG. 3 is a flowchart for component usage for recyclable and disposable components.

FIG. 4 is a flow chart for an alternate embodiment of using medical test kits in a medical kiosk.

FIG. 5 is a system components diagram.

FIG. 6A is a schematic view of a hardware test kit with an eyepiece used for retinal scans.

FIG. 6B is a schematic view of the use of the eyepiece shown in FIG. 6A.

FIG. 7A is a schematic view of a hardware test kit with a filter component for a digital camera.

FIG. 7B is a schematic showing use of the hardware test kit shown in FIG. 7A.

FIG. 8A is a perspective view of a hardware test kit with a portable digital camera.

FIG. 8B is a perspective view of an alternate digital camera.

FIG. 9 is a schematic view of a hardware test kit with a disposable skin sensor.

FIGS. 10A-C are schematic views of a sample collectors with a breath chambers.

FIGS. 11A-C are schematic views of embodiments of sample collectors with a whistle for breath collection.

FIG. 12 are schematic views of sample collectors for saliva collection.

FIG. 13 are perspective views of sample collectors for skin collection.

FIG. 14 are perspective views of sample collectors for hair collection.

FIG. 15 are perspective views for various health care item such as a healing with light bandage.

### DETAILED DESCRIPTION OF THE INVENTION

The present invention will be directed in particular to elements forming part of, or in cooperation more directly with, the apparatus in accordance with the present invention. It is to be understood that elements not specifically shown or described may take various forms well known to those skilled in the art.

The general method of the invention is the use of an unassisted medical kiosk to determine tests to be performed at the medical kiosk, dispense a test kit, perform said tests, reclaim and decontaminate the test kit, dispose of medical waste, and record the results of said tests.

Testing Procedures Integrated into a Medical Kiosk

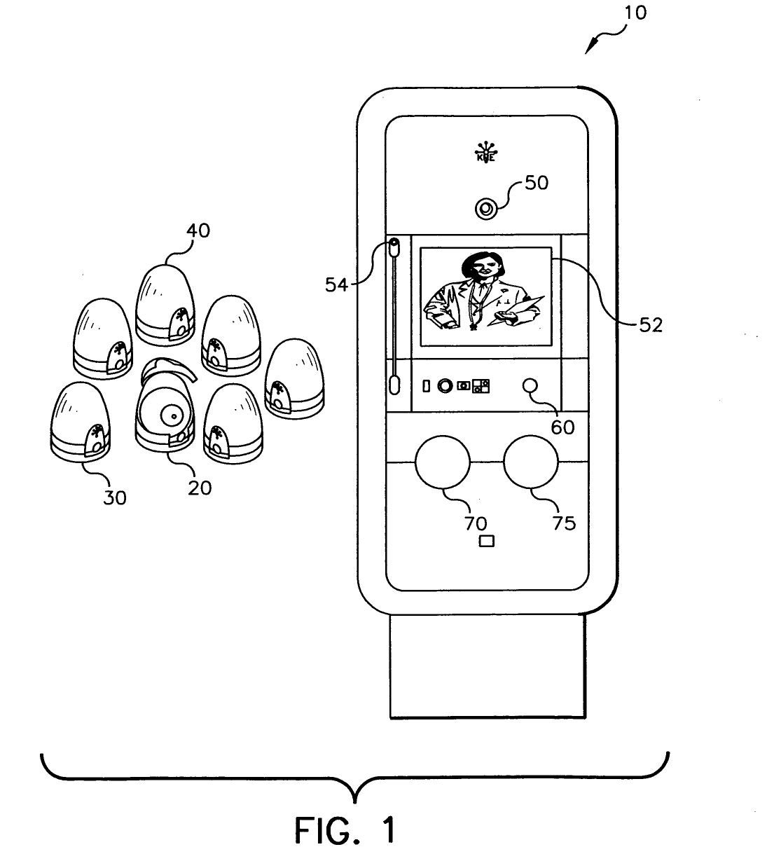

FIG. 1 illustrates a diverse set of medical testing procedures or test kits integrated into a medical kiosk 10.

Medical tests can include, but are not limited to, cholesterol, blood glucose, drug detection, Alzheimer's, pregnancy related tests, eye exam, saliva, blood, skin, bodily fluid, body image, electrocardiogram, hyperspectral imaging, multispectral imaging, or oxygenation. Medical test kits provided to a patient at the medical kiosk 10 contain components necessary to perform the medical test in an unassisted manner. These test kits are composed of recyclable and/or disposable components and fall into three categories:
Test hardware 20: includes custom test hardware used for a medical testing procedure. Lenses for use with a digital camera for capturing retinal scans and a wireless digital camera which patients can use either at the medical kiosk 10 or take home to capture an image are two examples of test hardware 20.
Sample collector 30: includes components used for capturing a sample (e.g. breath, saliva, hair) to be analyzed for the medical test.
Health care items 40: include consumable medical supplies or health care items 40. A special bandage for accelerating the healing of wounds is one example.

While most testing procedures involve the use of a medical test kit, some procedures are performed directly by the medical kiosk 10 without additional test kit components. One example is the use of a high resolution digital camera 54 integrated on a tethered arm to capture digital images for diagnostic purposes. For example, images from this digital camera 54 can be used to monitor the progress of wound care, analyze moles, or diagnose other skin conditions.

The medical kiosk 10 itself may contain a number of additional features:
A multi-purpose digital camera 50, is used for capturing a retinal scan for retinal analysis. Additionally this camera captures real time video to allow the patient to videoconference with a medical professional and/or support person at some remote location. It can also be used to confirm the identity of the person using the kiosk using existing image identification methodologies.
The display screen 52 presents important instructions, medical information, or test results from the session to the patient. To simplify the user interface, this display can incorporate a touch screen for use in navigating through the testing process. The display screen 52 can also show a live video feed of a physician, medical professional, or support person.
   Additionally, this display screen 52 could display downloaded images or video from some remote site.
   Systems Diagram and Potential Data Forms on EPR Embodiments

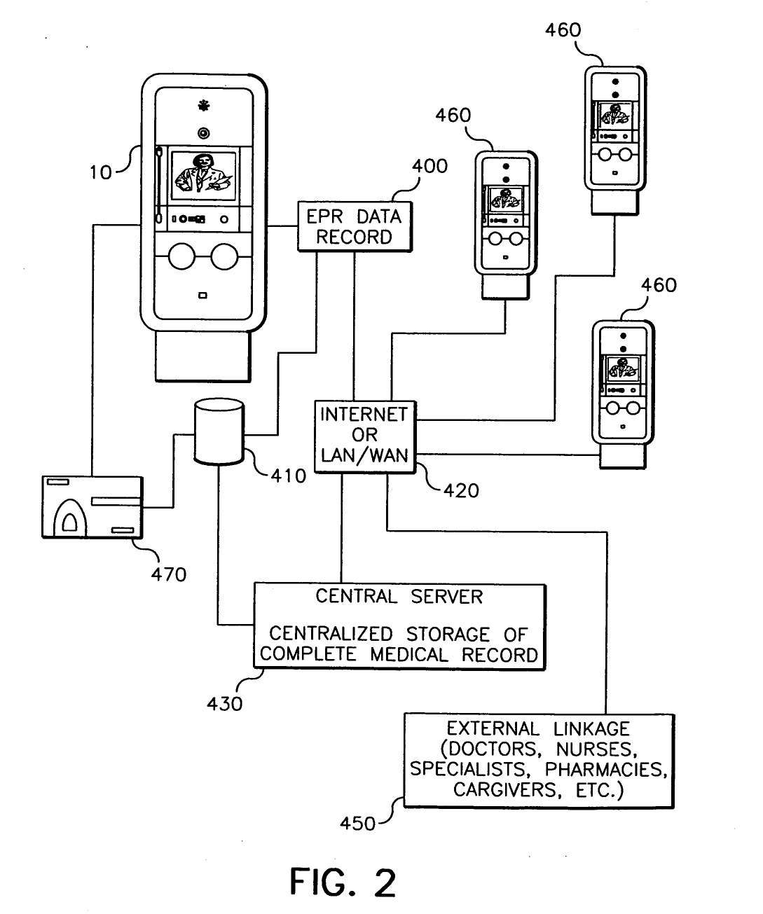

Referring now to FIG. 2, illustrating a system diagram for data exchange within a medical kiosk environment. The medical kiosk can be a standalone apparatus, or networked with other medical kiosks 460 and a central server. As disclosed in commonly-assigned co-pending U.S. patent application Ser. No. ______ (Attorney Docket No. 88970/NAB this system configuration allows the medical kiosk to access electronic patient records (EPR) in order to determine medical tests to be performed, and to update the EPR and communicate test results to interested third parties such as the patient's doctor, family members or insurance agency.

When a patient accesses a medical kiosk 10, the system will require authentication prior to performing a test procedure 100. The EPR medical access card 470 is an example of an embodiment used to validate and verify a patient's identity. Once validated, the medical kiosk 10 will attempt to access the EPR data record 400 for the patient. The EPR data record 400 contains: patient information, electronic patient records (EPR), current care plan, user preferences, permission levels for access to medical records, and physician or insurance company information. This EPR data record 400 can be stored on the EPR medical access card 470, on a local storage device 410 or from a central server 430 (accessible via the Internet or a LAN/WAN connection 420). If their care plan requires that specific tests be performed, the patient will be informed of the tests that need to be completed 110. Patients will have the option to select one of these tests, or they can choose to select another service 110. If no care plan exists, patients will have the option to choose what service they would like to perform 110. A standard credit card would be used as the initiation method.

Flowchart for Component Usage for Recyclable and Disposable Components

The general method for test kit handling includes: dispensing the test kit through the test kit dispenser 70, collecting the used test kit through test kit collector 75, sterilizing and recycling reusable test kit components, and disposing of non reusable test kit components.

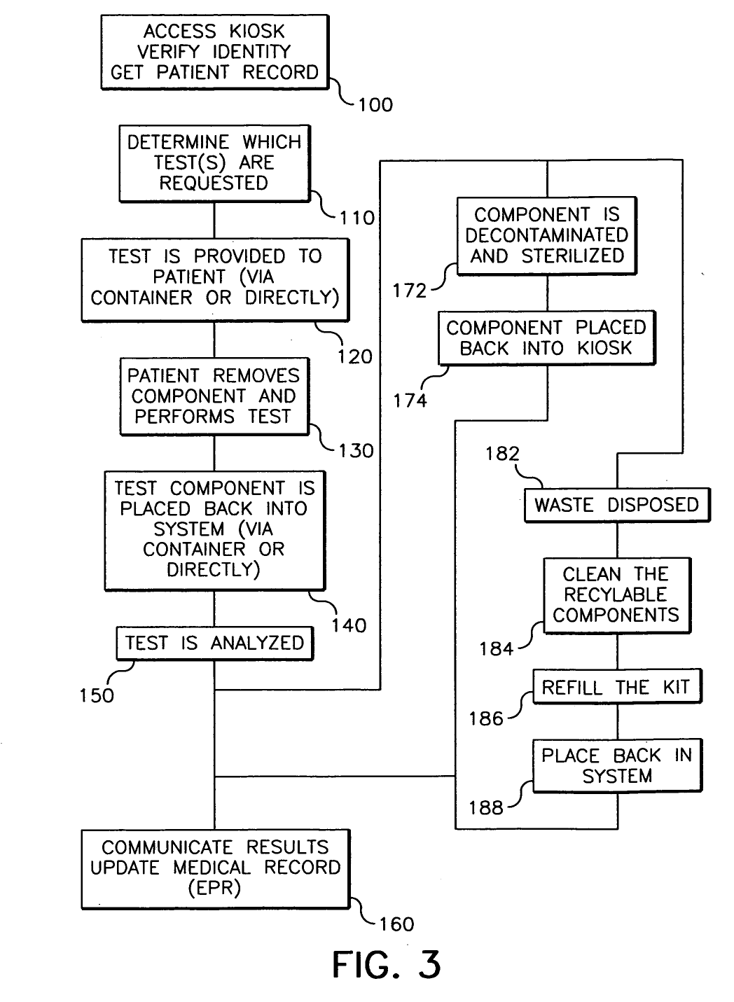

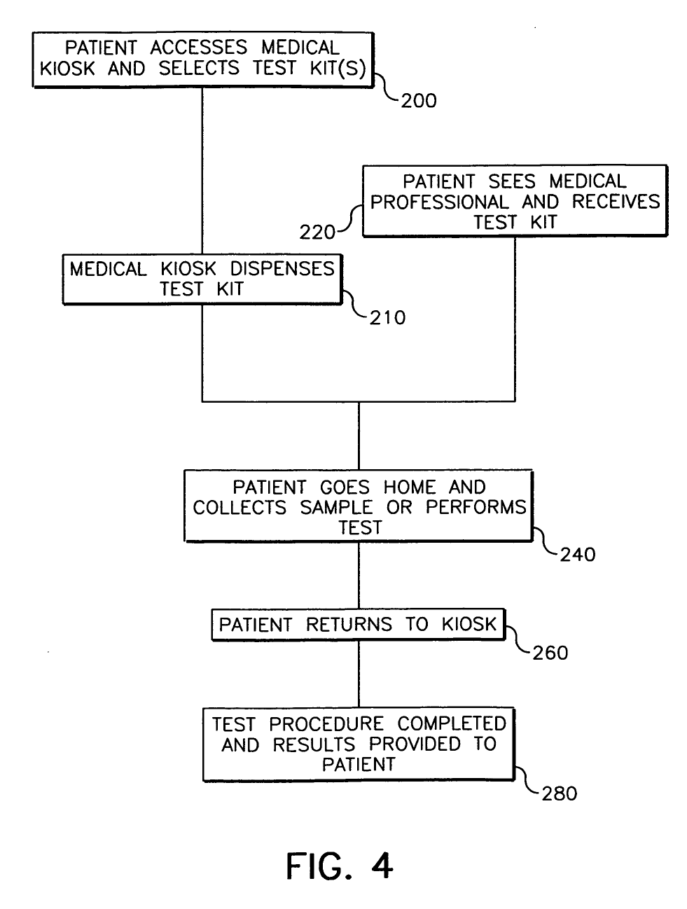

FIG. 3 illustrates handling recyclable and disposable components in a medical kiosk 10. Once the patient is identified and desired test(s) are determined, the medical kiosk 10 will automatically dispense 120 the appropriate custom medical test kit via the test kit dispenser 70. This kit may contain test hardware 20, a sample collector 30, or a health care item 40.

The patient removes the medical test kit from the test kit dispenser 70, and is instructed on how to perform the test procedure. These instructions are either presented on the display screen 52 or a videoconference may be established (video displayed on the screen 52) where a trained professional will walk the patient through the test procedure.

The patient performs the test 130. When the patient completes the test, they return all the completed equipment 140 into a test kit collector 75 on the front of the medical kiosk 10. The test will be analyzed 150 within the medical kiosk 10 or communicated to a remote site for analysis. During the analysis, the patient will be given either verbal or visual feedback on the progress of the procedure via the test in progress indicator 60 on the front of the medical kiosk 10.

Once the test is completed, the patient has a number of options regarding their test results 160. Specifically they can:
View the test results directly on the display screen 52;
Print out results to keep as a permanent record;
Choose to add the test results to their electronic patient record (EPR); and
Choose to make the test results accessible to other family members and to submit the test results to their physician, a specialist, or their insurance company 450.

Overall customer satisfaction and continued usage of medical kiosks 10 will require that all aspects of the system focus on maintaining strictly controlled sanitary conditions. All test kits and consumables will be reclaimed by the medical kiosk 10, via a test kit collector 75 on the front of the apparatus. This will ensure that the kiosk environment remains clean and uncluttered. Internally, the medical kiosk 10 will determine which parts are recyclable or disposable and process the parts according to strict sanitary guidelines.

Recyclable component pieces, in the form of reusable test hardware or sample chambers will be meticulously cleaned, disinfected and sterilized 172. For example, test components may be placed in an autoclave or washed with an approved chemical agent to sterilize component parts. Once the reusable test components have been cleaned and sterilized they will be installed in at least one additional medical test kit for later use 174. The recyclable components may be sterilized within the medical kiosk 10 or be removed by service personnel, sterilized at a remote site, and returned in a new medical test kit at a later date. The sterilization can be done within the kiosk via standard means, like an autoclave, and replaced into the system using a sorting system like those used to sort coins (since each component will have a unique shape and size). It may be more economical to have the sterilization done as a maintenance person is restocking the components.

Disposable items from test kits will be handled as medical waste. The medical kiosk 10 will contain one or multiple disposal bins for used consumables 182. Any items that need to be treated, as medical waste will follow approved guidelines for storage and disposal. These medical bins are designed in such a manner that unskilled professionals can service the medical kiosk 10. For example, the bins will be designed to prevent leakage or loss of used consumables similar to bins used to dispose of hypodermic needles. The system would allow for the entire bin to be replaced.

For test kits that contain both recyclable and non-reusable components the same procedures as described above will be followed. Non-reusable components will be disposed of as medical waste 182. Recyclable components will be cleaned 184, refitted with new consumable components 186, and installed in at least one additional medical test kit for later use 188. This can be done as a maintenance function or an automated system can be devised as a standard mechanical engineering exercise.

The medical kiosk 10 may also go through a cleaning cycle between patients. For example, the system may close a curtain and spray the exterior of the medical kiosk with a disinfecting agent. This can be done by a support person or automated.

Alternative Means that Test Kits can be Used in a Medical Kiosk

The entire cycle of determining medical tests to be performed, dispensing the medical test kits, performing and analyzing the test, and reporting the results does not need to occur entirely at the medical kiosk 10 or in one session at the medical kiosk.

In one alternative embodiment, the patient accesses a medical kiosk 10 and the required medical tests are determined 200. The medical kiosk 10 dispenses a test kit 210 along with instructions on how to use the kit. The patient ends the session, and goes to a remote site (such as their home), and collects the necessary sample 240. At some later point in time, the patient returns to a medical kiosk 260. Due to the network capability and EPR access illustrated in FIG. 2 the patient has the option of returning to the first medical kiosk or a second medical kiosk to complete the medical test. The medical test kit is collected through the test kit collector 75 and the medical kiosk completes the test procedure, and optionally updates the electronic patient record (EPR) and sends the results to a third party 280.

In a second alternative embodiment, the patient receives the medical test kit and instructions from a health care provider 220. The patient goes to a remote site and collects the necessary sample. At some later point in time, the patient returns the medical test kit to a medical kiosk 260 to complete the medical test. The medical test kit is collected through the test kit collector 75 and the medical kiosk completes the test procedure, and optionally updates the electronic patient record (EPR) and sends the results to a third party 280.

System Components Diagram

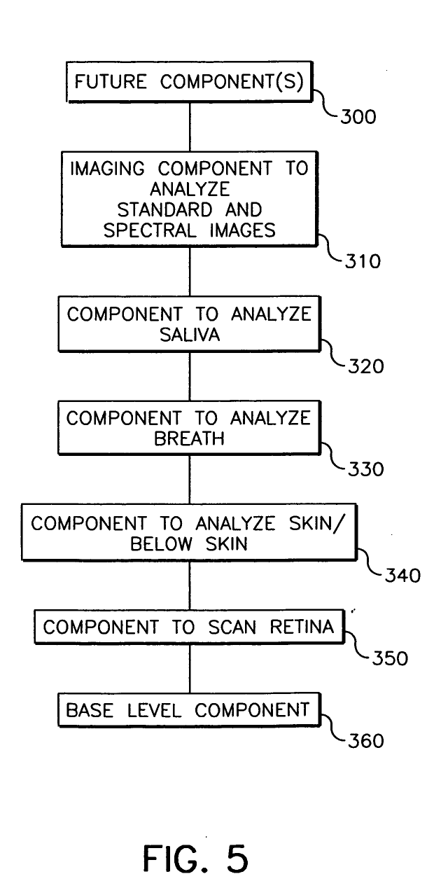

The present invention describes a medical kiosk 10 that is built upon a customizable framework as illustrated in FIG. 5.

The customizable framework has significant economic, size and ease-of-use advantages. Economically because components that are not used will not be part of the cost of the system, size of the kiosk 10 is determined by the number of components, especially when input and analysis tools may be needed and ease-of-use because learning new modalities and the number of choices that are available can be confusing.

The medical kiosk framework can be customized to best meet the needs of the following variables:
Differing markets—Elder care facilities, college campuses and primary schools have needs for different medical tests due to the demographics and culture of the residents in these environments.
Different locales—A mall location would need to perform tests more quickly and have less privacy than a small clinic or doctor's office.
Different levels of complexity.
Levels of assistance—Some kiosk facilities may require on-site support (in any one of a number of levels; clerk, technician, nurse, etc.) due to the nature of their clientele or location.
Customs—Some cultures may shun certain types of tests as being associated with lower class or certain tests may indicate a type of disease that is particularly difficult to contain and contagious.
Owner needs—The purchaser of the kiosk may only want certain tests performed (e.g. a SARS clinic may only want to test for diseases with high temperature, for example).

There are six specific medical testing components of the present invention:
Retinal or ocular scan and analysis 350
Breath collection and analysis 330
Saliva collection and analysis 320
Skin collection and analysis 340
Image collection and analysis 310

The architecture allows for anticipated additional components 300 to be added at a future time.

There is also a base level component 360 that has the basic components required for all of the embodiments of the kiosk. These functionalities are in addition to the basic functions of a standard personal computer such as a processor, storage, memory and the like (included on standard PCs today). Included in this base component are:
Blood pressure and base level cardiovascular tests
Software and hardware to be able to read and write patient records
Billing information
Connectivity to a central server/Internet
Premium services (like the ability to communicate to a doctor or a nurse for an additional fee, or transmission of images and records to a third party).
External and additional storage
Others as deemed necessary
   Hardware Test Kit—Eyepiece Used for Retinal Scans

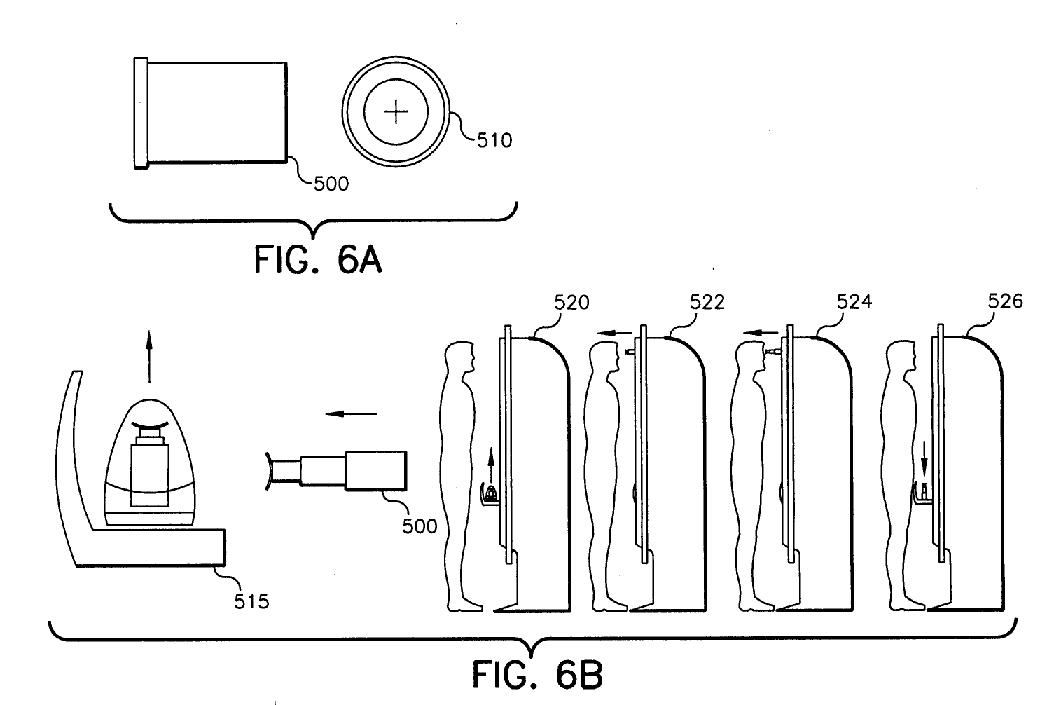

FIGS. 6A and 6B illustrate a system diagram for performing a retinal eye scan. In this scenario, the patient removes a medical test kit containing the eyepiece lens component 500 from the test kit dispenser 520. An embodiment of a retinal lens within a component delivery system 515 is shown. The medical kiosk display screen 52 instructs the patient to insert the eyepiece into a slot on the kiosk 522. This eyepiece could be inserted either in front of the digital camera 50 or be attached to the high resolution digital camera 52 on the tethered arm. The patient would be instructed to look into the eyepiece 524 and focus directly at the center of the target 510. A high-resolution image is captured of the retina. This image is analyzed locally on the medical kiosk 10 or is sent to some remote location and analyzed there. A test in progress indicator 60 informs the patient that the medical kiosk 10 is performing the ocular analysis. Analysis results are displayed on the screen 52 or printed out at the patient's request. Optionally, the results can be sent to a third party using secure e-mail.

Once the test is completed, the medical kiosk instructs the patient to place the eyepiece back in the test kit and return it to the test kit collector 526. The kit is retracted back into the medical kiosk 10. For disposable eyepieces, the medical kiosk 10 disposes of the test kit and eyepiece in an appropriate manner. For recyclable eyepieces, the medical kiosk decontaminates and sterilizes all the components and returns the medical test kit back into the system for later usage.

Hardware Test Kit—Filter Component for a Digital Camera

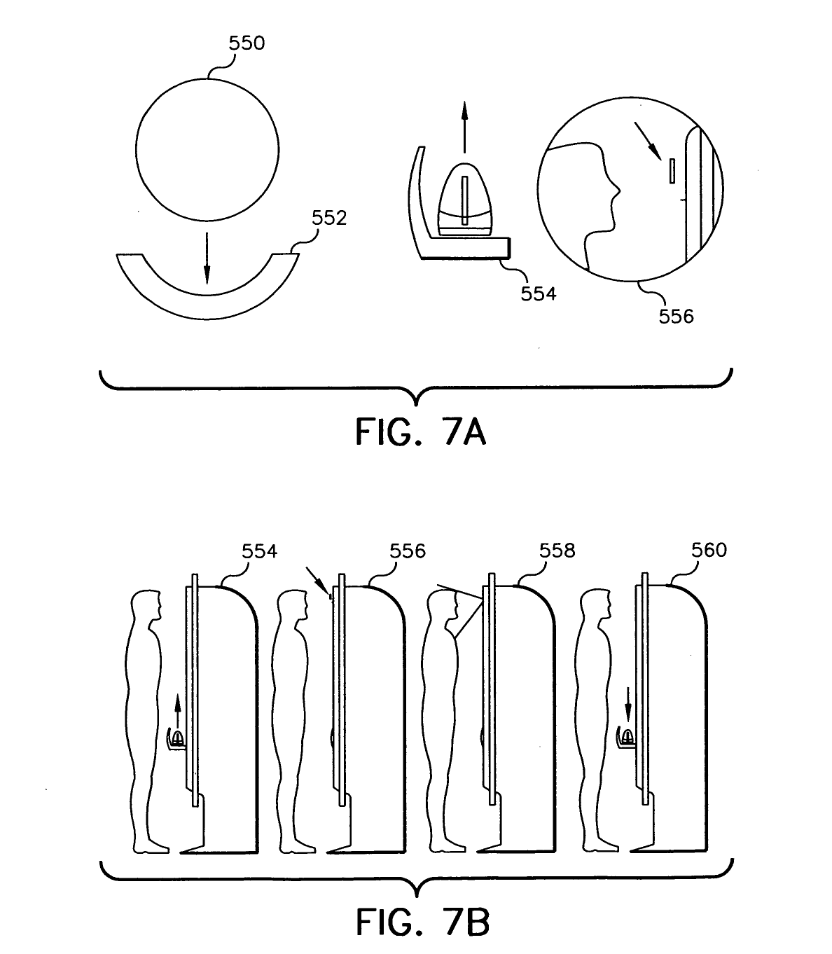

FIGS. 7A and 7B illustrate a system diagram for performing spectral imaging of the human body. Spectral imaging of human flesh enables analysis and diagnosis of wounds, infections and possible cancerous lesions. Filter components allow images to be captured in the near infrared or other parts of the electromagnetic spectrum.

In this scenario, the patient removes a test kit containing the filter component 550 from the test kit dispenser 554. The medical kiosk display screen 52 instructs the patient to insert the filter component 550 into a camera filter holder 552 on the kiosk. This filter component 550 could be inserted either in front of the digital camera 50 or be attached to the high resolution digital camera 54 on the tethered arm 556. The patient is instructed to place the skin sample to be analyzed directly in front to the digital camera. A high-resolution image is captured 558. This image is analyzed locally on the medical kiosk 10 or is sent to some remote location and analyzed there. A test in progress indicator 60 informs the patient that the medical kiosk 10 is performing the skin analysis. Analysis results are displayed on the screen 52 or printed out at the patient's request. Optionally, the results can be sent to a third party using secure e-mail.

Once the test is completed, the medical kiosk instructs the patient to place the filter component 550 back in the test kit and return it to the test kit collector 560. The kit is retracted back into the medical kiosk 10. For disposable filter components 550, the medical kiosk disposes of the medical test kit and filter in an appropriate manner. For recyclable filters, the medical kiosk decontaminates and sterilizes all the components and returns the medical test kit back into the system for later usage.

Hardware Test Kit—Portable Digital Camera

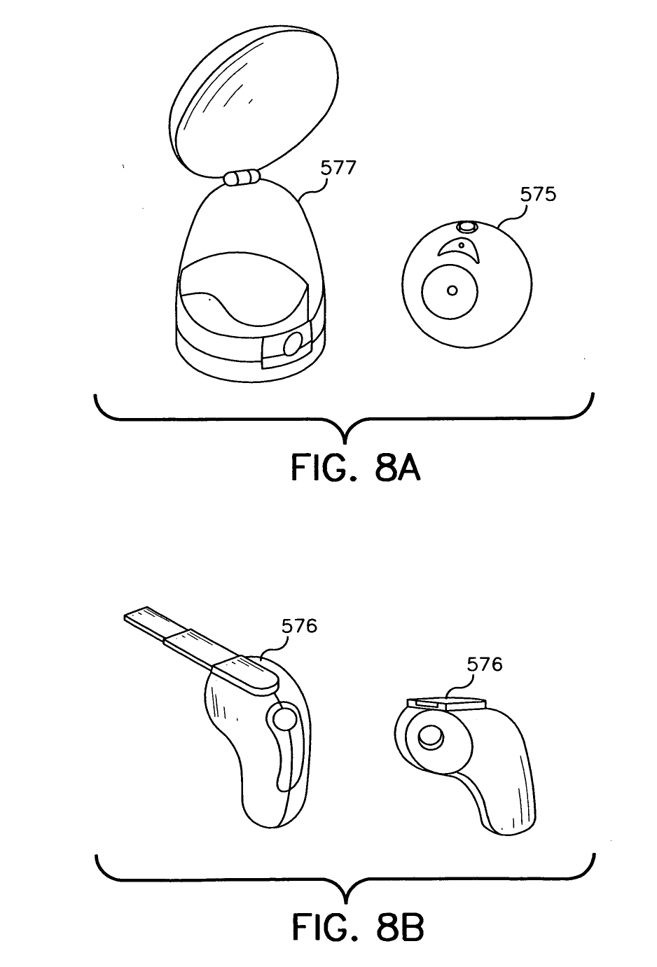

FIGS. 8A and 8B illustrate a portable digital camera 575 test kit delivered in container 577 from the kiosk. This portable digital camera offers a great deal of flexibility for capturing images. For example, a portable digital camera 575 could be used with existing medical kiosks 10 to take images of regions of the body that could not be viewed using the on board digital cameras 50, 54. Medical kiosks 10 could also be designed without any onboard digital camera 50, 54. This portable digital camera test kit 575 enable patients to capture images, perform retinal eye scans or do a spectral analysis of a skin sample or wound care (using optional spectral filters). Portable digital cameras could be physically tethered to the medical kiosk 10 or communicate with the kiosk using a wireless protocol (e.g. Bluetooth, RF).

Portable digital cameras 575 could also be taken to a remote site where a patient takes a picture of their skin or wound, in private. In this scenario the portable digital camera would be battery powered and contain on-board memory to store the image. At a later time, the patient returns to any medical kiosk 10 on the network to complete the test procedure.

Another embodiment of a portable digital camera is with a telescoping lens 576 that can be used to obtain a close-up view or even perform a macro image on contact with the skin.

Portable digital cameras 575 could be either disposable or recyclable. For recyclable digital cameras, the medical kiosk 10 decontaminates and sterilizes all components in the camera kit, and places the kit back into the kiosk for later use. Disposable digital camera components are discarded using approved methodologies.

Analysis results are displayed on the display screen 52 or printed out at the patient's request. Optionally, the results can be sent to a third party using secure e-mail.

Hardware Test Kit—Disposable Skin Sensor

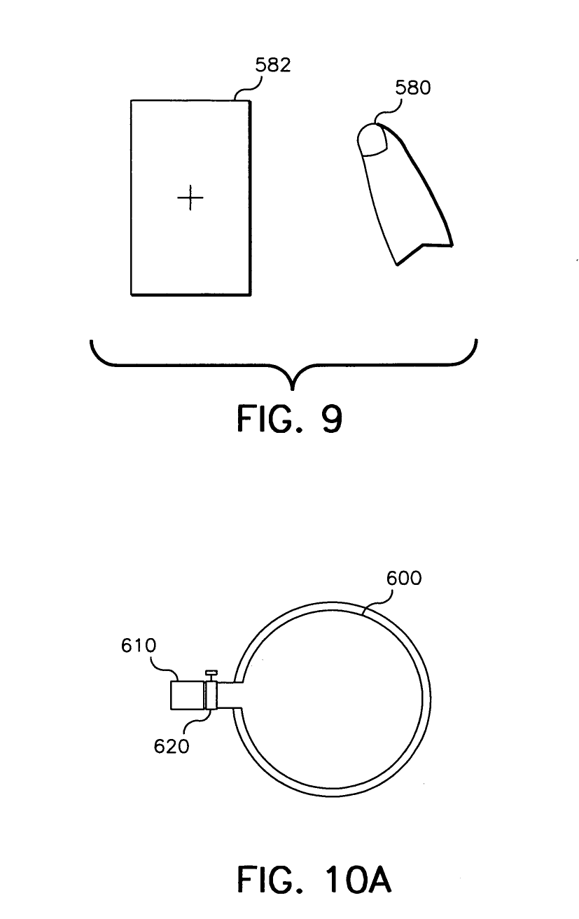

FIG. 9 illustrates a disposable skin sensor 580. In this scenario, the patient removes a test kit containing the disposable skin sensor 580 from the test kit dispenser 70. The medical kiosk display screen 52 instructs the patient to put on the disposable finger cot or glove 580 and place the skin sensor against the touch pad 582 on the front of the medical kiosk 10. The touch pad 582 initiates communication the skin sensor 580 to measure skin temperature or skin moisture. The touch pad 582 may also be a light source for spectroscopic analysis or provide a small current for galvanic analysis. This information is received and analyzed locally on the medical kiosk 10 or is sent to some remote location and analyzed there. A test in progress indicator 60 informs the patient that the medical kiosk 10 is performing the skin analysis. Analysis results are displayed on the screen 52 or printed out at the patient's request. Optionally, the results can be sent to a third party using secure e-mail.

Once the test is completed, the medical kiosk instructs the patient to place the skin sensor 580 back in the medical test kit and return it to the test kit collector 75. The kit is retracted back into the medical kiosk 10, and disposed of in an appropriate manner.

Sample Collector—Breath Chambers

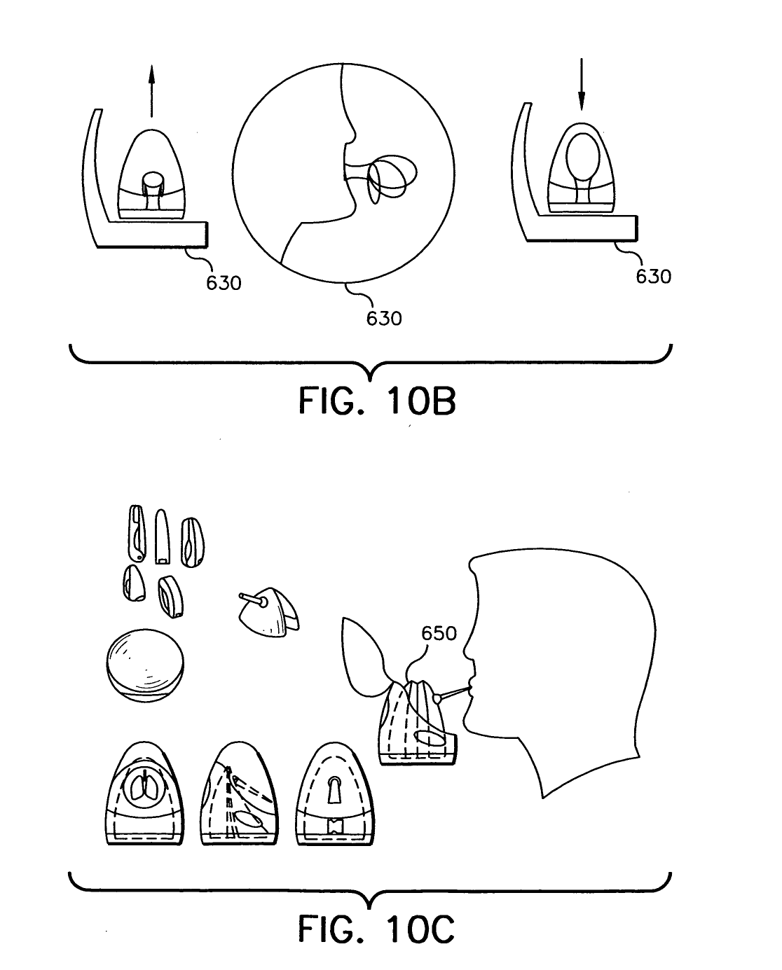

FIGS. 10A-10C illustrate embodiments for collecting breath samples. The collection balloon 600 represents a recyclable embodiment where the patient opens a valve 620 and blows into the mouthpiece 610. The collection balloon 600 fills with air and is returned to the medical kiosk 10 for analysis. The medical kiosk 10 removes and disposes of the detachable mouthpiece. The collection balloon 600 is either disposed of or decontaminated and sterilized prior to being placed back in the kiosk for later use.

The second embodiment shows a disposable breath chamber 650 that a patient blows into. A tab pops up once the chamber is full to indicate that the chamber is full. Once the analysis is completed the breath chamber is disposed of within the medical kiosk 10.

A method by which these embodiments can be delivered to the patient and returned to the kiosk 630 is shown.

Analysis of blood glucose in a breath sample is an example of this type of breath test. For this scenario, the medical kiosk 10 dispenses the medical test kit via a test kit dispenser 70. The patient removes the kit and follows the instructions presented on the display screen 52. The patient breathes into the sample chamber (either the collection balloon 600 or expandable breath chamber 650). The breath sample chamber is then placed in the test kit receptor 75. The medical kiosk 10 reclaims the kit and extracts air from the collection balloon 600, or expandable breath chamber 650, and analyzes the sample. A test in progress indicator 60 informs the patient that the medical kiosk 10 is performing the breath analysis. Analysis results are displayed on the screen 52 or printed out at the patient's request. Optionally, the results can be sent to a third party using secure e-mail.

Sample Collector—Whistle Embodiment for Breath Collection

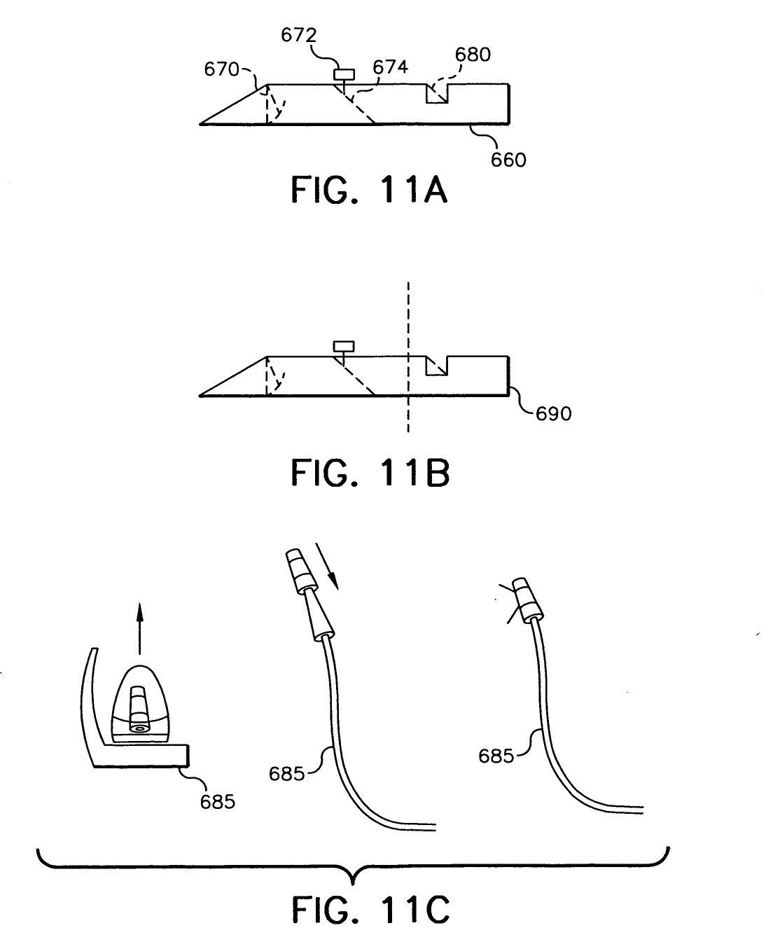

FIGS. 11A-11C illustrate another embodiment for collecting a breath sample. The breath whistle 660 represents a disposable embodiment in which the patient blows into a whistle. As the patient blows, air pressure forces both the front air valve 670 and rear air valve 674 open, and escaping air 680 makes a whistling sound. When the patient stops blowing, both the front air valve 670 and rear air valve 674 snap shut, thus capturing a air sample. The whistle is inserted into a slot on the front of the kiosk to deliver the breath sample when the release valve 672 is depressed. The medical kiosk 10 analyzes the sample. A test in progress indicator 60 informs the patient that the medical kiosk 10 is performing the breath analysis. Analysis results are displayed on the display screen 52 or printed out at the patient's request. Optionally, the results can be sent to a third party using secure e-mail.

Another embodiment is a whistle connected to a tube connected to the kiosk 10 whereby a disposable cover is placed on the whistle and a sound is emitted when a large enough sample is collected 685.

Once the test is completed, the medical kiosk instructs the patient to place the whistle 550 back in the test kit and return it to the test kit collector 75. The kit is retracted back into the medical kiosk 10, and disposed of in an appropriate manner 690.

Sample Collector—Saliva Collection

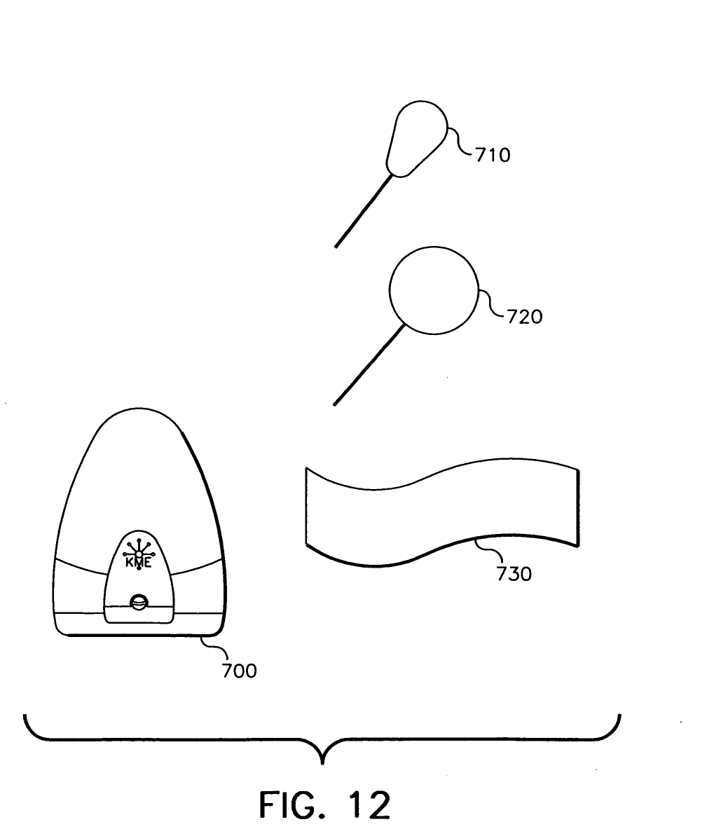

FIG. 12 illustrates an embodiment for collecting a saliva sample. In this scenario the patient opens the medical test kit 700 and removes a saliva collection device. A mouth swab 710, lollipop 720, and chewing gum 730 represent three such collection methods. The mouth swab is used to swab the mouth or throat to collect a saliva sample or throat culture. The lollipop contains a porous surface where saliva would be absorbed into the interior of the sphere. The chewing gum would also absorb saliva. In each of these scenarios, the sample collectors are placed back into the medical test kit 700 and re-inserted in the test kit collector 75 where the medical kiosk would remove these embodiments, analyze the sample and dispose of the collection device in an appropriate manner. A test in progress indicator 60 informs the patient that the medical kiosk 10 is performing the saliva analysis. Analysis results are displayed on the display screen 52 or printed out at the patient's request. Optionally, the results can be sent to a third party using secure e-mail.

Sample Collector—Skin Collection

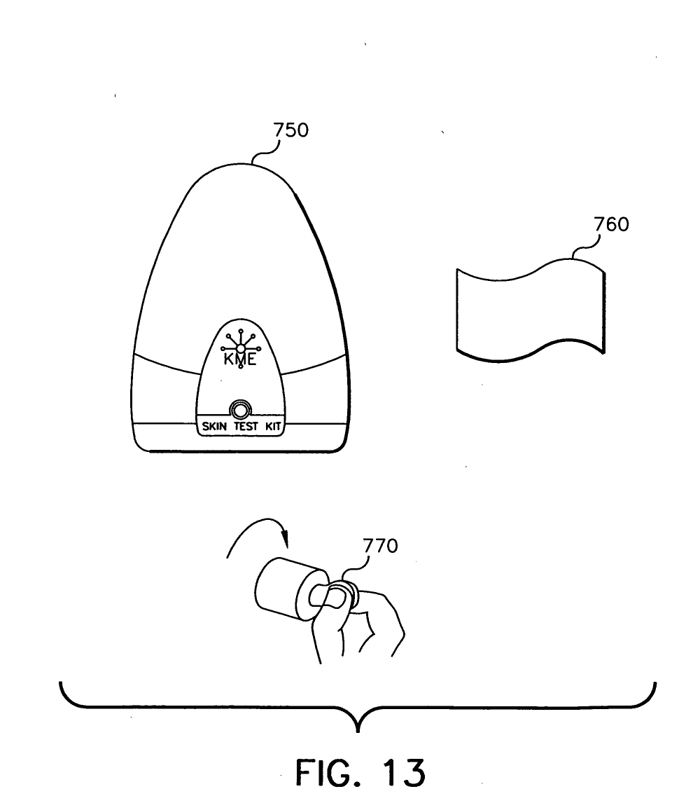

FIG. 13 illustrates an embodiment for collecting a skin sample. Skin samples have been shown to provide information in the areas of pulse oximetry, optical coherence tomography, fluorescence imaging, laser Doppler flowmetry, photoplethysmography, thermography, infrared spectroscopy, photoacoustic spectroscopy, Raman spectroscopy, electrochemical impedance spectroscopy, laser speckle imaging, electrocardiography, echocardiography, electromyography, confocal microscopy, and multiphoton microscopy. In this scenario, the patient opens the medical test kit 750 and removes an abrasive cloth 760. The patient is instructed to rub this cloth against their skin to remove a few skin cells, which adhere to the cloth. The abrasive cloth is embodied as a rolled up collector 770 and placed back into the medical test kit 750 and reinserted back into the medical kiosk 10 for analysis and disposal. A test in progress indicator 60 informs the patient that the medical kiosk 10 is performing the skin analysis. Analysis results are displayed on the screen 52 or printed out at the patient's request. Optionally, the results can be sent to a third party using secure e-mail.

Sample Collection—Hair Collector

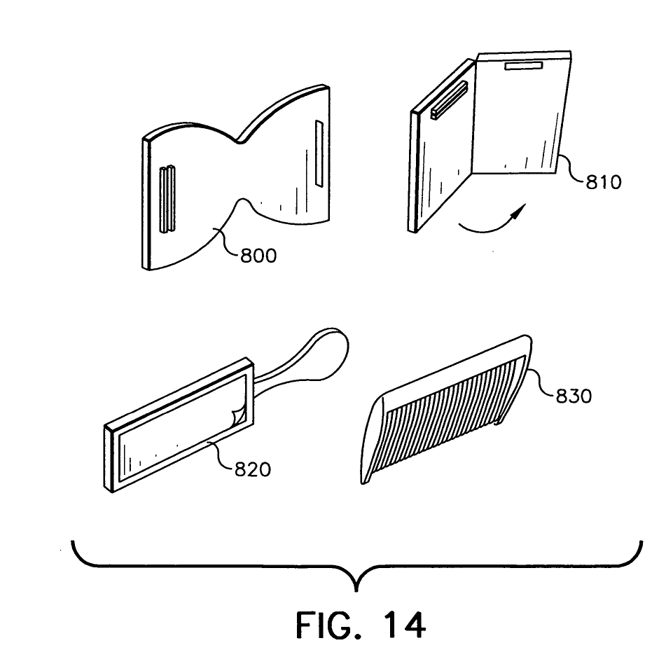

FIG. 14 illustrates how a hair sample can be introduced into a kiosk. In addition to DNA information, hair contains information about drug usage and dosages which would be a useful test at a kiosk. Four possible embodiments of hair collection methodologies are shown. The first two methodologies show a “book” type approach 800 and 810 which would grab and secure the hair and then allow it to be placed in the kiosk for analysis. A third embodiment shows an adhesive attached to a handle 820 that can be placed on the hair and pulled away leaving a sample. Another embodiment is with a simple comb 830 with an dense array of teeth that would allow a sample to be withdrawn.

Analysis results are displayed on the display screen 52 or printed out at the patient's request. Optionally, the results can be sent to a third party using secure e-mail.

Health Care Item—Healing With Light Bandage

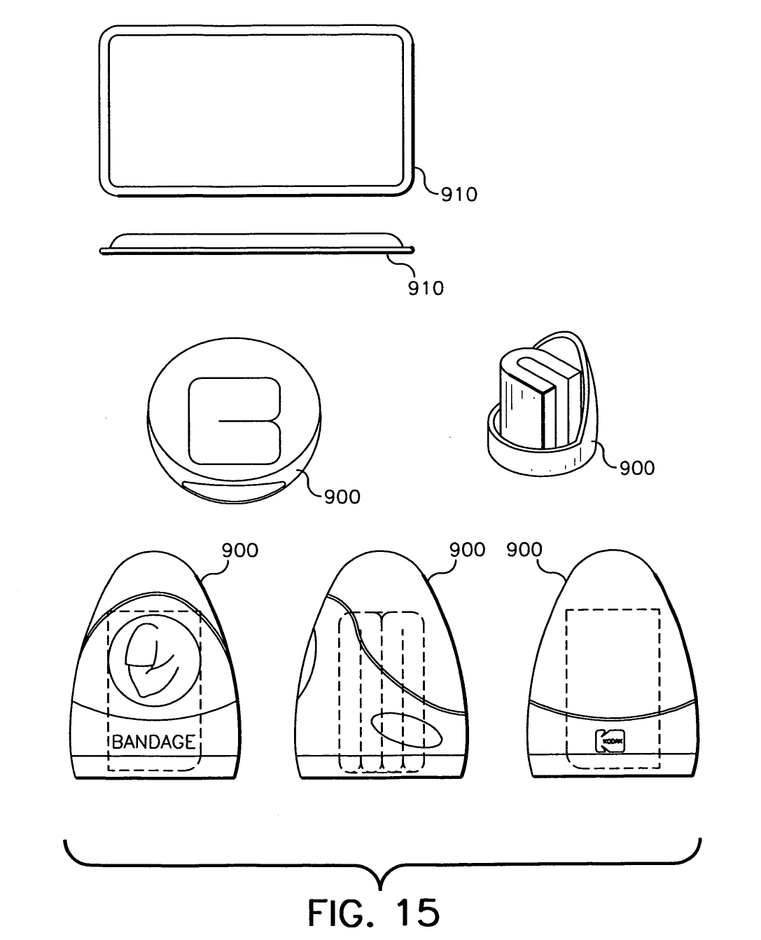

FIG. 15 illustrates a test kit 900 that contains a special bandage 910 used to accelerate the healing of wounds. In one scenario, the patient removes the bandage from the kit and places it over a wound. Once opened, the bandage begins to emit light, which speeds the healing process. The patient is able to leave the kiosk and the bandage continues to provide healing light to the wound for a number of hours. The patient removes this bandage at home at a later time and disposes of it along with their garbage.

In a second scenario, the bandage contains electrodes that are inserted into the medial kiosk. When the kiosk applies a signal to the electrodes, the bandage emits a healing light. In this scenario, the patient remains in the kiosk for 5-20 minutes. Once the session is completed, the patient is instructed to place the bandage 910 back in the test kit 900, and return it to the test kit collector 75. The kit is retracted back into the medical kiosk 10 and disposed of in an appropriate manner.

### PARTS LIST

10 medical kiosk
20 test hardware
30 sample collector
40 health care items
50 digital camera
52 display screen/interaction area
54 high resolution digital camera for evaluating conditions
60 test in progress indicator
70 test kit dispenser
75 test kit collector
100 start session (verify patient identity and access electronic patient records)
110 determine which tests are required
120 test kit is provided to patient
130 patient removes components and perform test
140 test component place back into medical kiosk
150 test is analyzed
160 communicate results and update medical records
172 components decontaminated and sterilized
174 test kit place back in system
182 disposable items disposed of as medical waste
184 optional recyclable components decontaminated and sterilized
186 refill kit with new consumable items
188 place kit back into system
200 patient access medical kiosk and selects test procedure
210 medical kiosk dispenses test kit
220 patient sees medical professional and receives test kit
240 patient goes home (collects sample or performs test)
260 returns to medical kiosk
280 test procedure completed and results provided to patient
300 future components
310 imaging components to analyze standard and spectral images
320 component to analyze saliva
330 component to breath
340 component to analyze skin
350 component to scan retina
360 base level component
400 EPR data record
410 medical kiosk local storage
420 Internet or LAN/WAN
430 central server
450 external linkages
460 secondary medical kiosk
470 EPR medical access card
500 eyepiece lens component
510 target indicator in eyepiece
515 retinal lens in a test kit
520 medical kiosk dispenses test kit with eyepiece
522 eyepiece inserted into slot on medical kiosk
524 patient looks into eyepiece
526 eyepiece reclaimed in medical test kit collector
550 filter component for a digital camera
552 camera filter holder
554 medical kiosk dispenses test kit with eyepiece
556 filter inserted into slot on medical kiosk
558 patient is imaged
560 filter component reclaimed in medical test kit collector
575 portable digital camera
576 camera with telescoping lens
577 container for recyclable camera
580 disposable finger cot or glove
582 touch pad on medical kiosk
600 collection balloon
610 detachable mouthpiece
620 valve
630 embodiment within a test kit
650 expandable breath chamber
660 breath whistle
670 front air valve
672 release valve
674 rear air valve
680 air escape
685 tethered whistle
690 whistle inserts into medical kiosk
700 saliva test kit
710 mouth swab
720 lollipop
730 chewing gum
750 skin sample test kit
760 abrasive cloth
770 rolled adhesive cloth
800 hair collection book 1
810 hair collection book 2
820 hair collection adhesive
830 hair collection comb
900 healing with light test kit
910 bandage

1. A method of providing medical services through a kiosk comprising:
determining medical test to be performed;
dispensing a test kit;
performing said tests;
decontaminating said the test kit; and
recording the results of said tests.

2. A method as in claim 1 wherein said medical tests are selected from a group comprising a saliva analyzer, a breath analyzer, a skin analyzer, and ocular analyzer.

3. A method as in claim 1 including the additional step of disposing of medical waste.

4. A method as in claim 1 including the additional step of reporting said results.

5. A method of distributing and collecting medical test kits in a medical kiosk comprising:
dispensing said test kit;
collecting the used test kit;
sterilizing reusable components of said kit; and
recycling said reusable components of said kit.

6. A method as in claim 5 comprising the additional step of:
disposing of non reusable components of said kit.

7. A method as in claim 5 comprising the additional step of:
installing said reusable components into at least one additional medical test kit.

8. A method of distributing and collecting medical test kits in a medical kiosk comprising:
dispensing said test kit;
collecting the used test kit; and
disposing of non reusable components of said kit.

9. A method of providing medical services through a kiosk comprising:
determining medical test to be performed;
dispensing a medical test kit to a patient from a first medical kiosk;
removing said medical test kit to a remote location;
collecting a sample with said medical test kit; and
returning said medical test kit to said first kiosk or a second kiosk.

10. A method as in claim 9 comprising the additional step of:
decontaminating said the test kit.

11. A method as in claim 9 comprising the additional step of:
performing medical tests at said first or second medical kiosk.

12. A method as in claim 9 comprising the additional step of:
recording the results of said medical tests.

13. A method of providing medical services through a kiosk comprising:
dispensing a medical test kit to a patient from a health-care provider;
removing said medical test kit to a remote location;
collecting a sample with said medical test kit; and
returning said medical test kit to a medical kiosk.

14. A method as in claim 13 comprising the additional step of:
decontaminating said the test kit.

15. A method as in claim 13 comprising the additional step of:
performing medical tests at said first or second medical kiosk.

16. A method as in claim 13 comprising the additional step of:
recording the results of said medical tests.

17. A method of providing medical services at medical kiosk comprising:
dispensing recyclable or disposable non-invasive medical test kits;
performing test procedures using onboard instrumentation or said medical test kit;
analyzing test results;
providing status information or a diagnosis;
reporting said test results;
reclaiming said medical test kits;
disposing of medical waste;
decontaminating said medical kiosk; and
sterilizing reusable components of said medical test kit.

18. A method as in claim 17 comprising the additional step of:
dispensing basic health care items.

19. (canceled)

20. (canceled)
